(19) United States
(12) Patent Application Publication
(10) Pub. No.: US 2002/0068709 A1
(43) Pub. Date: Jun. 6, 2002

(54) Therapeutic uses of LNA-modified oligonucleotides

(76) Inventors: Henrik Orum, Vaerlose (DK); Troels Koch, Copenhagen (DK); Jan Skouv, Espergade (DK); Mogens Havsteen Jakobsen, Vanlose (DK)

Correspondence Address: Dike, Bronstein, Roberts & Cushman, Intellectual Property Practice Group, Edwards & Angell, LLP, 130 Water Street, Boston, MA, 02109, US

(21) Appl. No.: 09/747,913

(22) Filed: Dec. 22, 2000

Related U.S. Application Data
(60) Provisional application No. 60/171,873, filed on Dec. 23, 1999.

(51) Int. Cl. A61K 48/00; C12N 15/87

(52) U.S. Cl. 514/44; 435/455

(57) ABSTRACT

The invention relates to therapeutic applications of LNA-modified oligonucleotides. In particular, the invention provides methods for treatment of undesired cell growth as well as treatment of inflammatory related diseases and disorders. Preferably, administration of an LNA-modified oligonucleotide modulates expression of a targeted gene associated with the undesired cell growth or an inflammatory related disease or disorder.

This application claims the benefit of U.S. Provisional Application Serial No. 60/171,873 filed Dec. 23, 1999, the teaching of which is incorporated herein by reference.

### BACKGROUND OF THE INVENTION

1. Field of the Invention

The invention relates to therapeutic applications of LNA-modified oligonucleotides. In particular, the invention provides methods for treatment of undesired cell growth as well as treatment of inflammatory related diseases and disorders. Preferably, administration of an LNA-modified oligonucleotide modulates expression of a targeted gene associated with the undesired cell growth or inflammatory related disease or disorder. That is, preferred use of LNA-modified oligonucleotide provides an antisense-type therapy with selective modulation of gene expression of predetermined targets.

2. Background

Certain nucleotide-based compounds have been utilized in various therapeutic applications. In particular, various oligonucleotides have been investigated including single stranded and double stranded oligonucleotides, and analogues. To be useful in in vivo applications an oligonucleotide must have a plethora of properties including the ability to penetrate a cell membrane, have good resistance to extra- and intracellular nucleases, have high affinity and specificity for the target and preferably have the ability to recruit endogenous enzymes such as RNAseH.

A fundamental property of oligonucleotides that underlies many of their potential therapeutic applications is their ability to recognise and hybridise specifically to complementary single stranded nucleic acids employing either Watson-Crick hydrogen bonding (A-T and G-C) or other hydrogen bonding schemes such as the Hoogsteen/reverse Hoogsteen mode. Affinity and specificity are properties commonly employed to characterise hybridisation properties of a particular oligonucleotide. Affinity is a measure of the binding strength of the oligonucleotide to its complementary target (expressed as the thermostability (Tₘ) of the duplex). Each nucleobase pair in the duplex adds to the thermostability and thus affinity increases with increasing size (No. of nucleobases) of the oligonucleotide. Specificity is a measure of the ability of the oligonucleotide to discriminate between a fully complementary and a mismatched target sequence. In other word, specificity is a measure of the loss of affinity associated with mismatched nucleobase pairs in the target.

Certain conformational restriction has been applied in recent years to oligonucleotides in the search for analogues displaying improved hybridisation properties compared to unmodified (2′-deoxy)oligonucleotides. For instance, there have been reported bicyclo[3.3.0]nucleosides with an additional C-3′, C-5′-ethano-bridge (see e.g., M. Tarköy et al., Helv. Chim. Acta, 1993, 76, 481); bicarbocyclo[3.1.0]nucleosides with an additional C-1′, C-6′- or C-6′, C-4′methano bridge (see e.g., K.-H. Altmann et al., Tetrahedron Lett., 1994, 35, 2331); bicyclo[3.3.0]- and [4.3.0] nucleosides containing an additional C-2′, C-3′-dioxalane ring synthesised as a dimer with an unmodified nucleoside where the additional ring is part of the internucleoside linkage replacing a natural phosphordiester linkage (see e.g., R. J. Jones et al., J. Am. Chem. Soc., 1993, 115, 9816); dimers containing a bicyclo[3.1.0]nucleoside with a C-2′, C-3′-methano bridge as part of amide- and sulfonamide-type internucleoside linkages (see e.g., C. G. Yannopoulus et al., Synlett, 1997, 378); bicyclo[3.3.0] glucose-derived nucleoside analogue incorporated in the middle of a trimer through formacetal internucleoside linkages (see e.g., C. G. Yannopoulus et al., Synlett, 1997, 378); tricyclo-DNA in which two five membered rings and one three membered ring constitute the backbone (see R. Steffens & C. J. Leumann, J. Am. Chem. Soc, 1997, 119, 11548-49); 1,5-Anhydrohexitol nucleic acids (see Aerschot et al., Angew. Chem. Int. Ed. Engl. 1995, 34(129 1338-39); and bicyclic[4.3.0]- and [3.3.0] nucleosides with additional C-2′, C-3′-connected six and five-membered ring; (see e.g., P. Nielsen et al., XII International Roundtable: Nucleosides, Nucleotides and Their Biological Applications, La Jolla, Calif., Sep. 15-19, 1996, Poster PPI 43). However, oligonucleotides comprising these analogues form in most cases less stable duplexes with complementary nucleic acids compared to the unmodified oligonucleotides.

Recently, novel DNA compounds referred to as Locked Nucleic Acids (LNA) have been reported (see International Patent Application WO 99/14226; P. Nielsen et al, J. Chem. Soc., Perkin Trans. 1, 1997, 3423; P. Nielsen et al., Chem. Commun., 1997, 9, 825; N. K. Christensen et al., J. Am. Chem. Soc., 1998, 120, 5458; A. A. Koshlin et al., J. Org. Chem., 1998, 63, 2778; A. A Koshkin et al. J. Am. Chem. Soc. 1998, 120, 13252-53; Kumar et al. Bioorg, & Med. Chem. Lett., 1998, 8, 2219-2222; and S. Obika et al., Bioorg. Med. Chem. Lett., 1999, 515). Interestingly, incorporation of LNA monomers containing a 2′-O, 4′-C-methylene bridge into an oligonucleotide sequence led to an unprecedented improvement in the hybridisation stability of the modified oligonucleotide (see above and e.g., S. K. Singh et al., Chem. Commun., 1998, 455). Oligonucleotides comprising the 2′-O, 4′-C-methylene bridge (LNA) monomers and also the corresponding 2′-thio-LNA (thio-LNA), 2′-HN-LNA (amino-LNA), and 2′-N(R)-LNA (amino-R-LNA) analogue, form duplexes with complementary DNA and RNA with thermal stabilities not previously observed for bi- and tricyclic nucleosides modified oligonucleotides. The increase in Tₘ per modification varies from +3 to +11° C., and furthermore, the selectivity is also improved. No other DNA analogue has reproducibly shown such high affinity for nucleic acids.

Molecular strategies are being developed to modulate unwanted gene expression that either directly causes, participates in or aggravates a disease state. One such strategy involves inhibiting gene expression with oligonucleotides complementary in sequence to the messenger RNA of a deleterious target gene. The messenger RNA strand is a copy of the coding DNA strand and is therefore, as the DNA strand, called the sense strand. Oligonucleotides that hybridise to the sense strand are called antisense oligonucleotides. Binding of these strands to mRNA interferes with the translation process and consequently with gene expression. For instance antisense oligonucleotides have been used as anti-cancer agents by targeting, and down regulating, the activity of various oncogenes or proto-oncogenes. See e.g., U.S. Pat. No. 5,098,890 (MYB antisense for treating hematologic neoplasms, including use in bone marrow purging); International Patent Application WO 91/93260 (ABL antisense for treating myeloproliferative disorders); International Patent Application WO 92/19252 and Ratajczak et al., Proc. Natl. Acad. Sci. USA 89, 1710-1714 (1992) (KIT for inhibiting malignant hematopoietic cell proliferation); International Patent Application WO92/20348 and Melani et al., Cancer Res. 51; 2897-2901 (1991) (MYB antisense for inhibiting proliferation of colon cancer cells); international Patent Application WO93/09789 (MYB antisense for inhibiting malignant melanoma cell proliferation); International Patent Application WO92/22303 and Szcylick et al., Science 253, 562-565 (1991) (BCR-ABL antisense for inhibiting leukemia cell proliferation); and U.S. Pat. No. 5,087,617 which describes bone marrow purging and in vivo therapy using antisense oligonucleotides to a variety of oncogenes or proto-oncogenes.

### SUMMARY OF THE INVENTION

The present invention provides use of LNA-modified oligonucleotides for treatment of undesired cell growth (i.e. cancer therapies) as well as for treatment of diseases and disorders associated with inflammation.

Preferably, an LNA-modified oligonucleotide is employed that enables effective modulation of a specific gene(s). As such the invention provides means to develop drugs against any human disease that are caused by either inherited or acquired genetic disorders, diseases in which a normal gene product is involved in a pathophysiological process or diseases that stems from the presence of infectious agents.

The invention may be used against protein coding genes as well as non-protein coding genes. Examples of non-protein coding genes include genes that encode ribosomal RNAs, transfer RNAs, small nuclear RNAs, small cytoplasmic RNAs, telomerase RNA, RNA molecules involved in DNA replication, chromosomal rearrangement of for instance immunoglobulin genes, etc.

According to one preferred embodiment of the invention, the LNA-modified antisense oligonucleotide is specific for cancer causing genes such as for instance the genes listed in table 1 below.

According to another preferred embodiment of the invention, the LNA-modified antisense oligonucleotide is specific for genes involved in inflammatory/allergic diseases such as for instance the genes listed in any one of tables 2 through 5 below.

The invention in general provides a method for treating diseases which are caused by expression of a normally unexpressed gene, abnormal expression of a normally expressed gene or expression of an abnormal gene comprising administering to a patient in need of such treatment an effective amount of an LNA-modified antisense oligonucleotide, or a cocktail of different LNA-modified antisense oligonucleotides, or a cocktail of different LNA-modified and unmodified antisense oligonucleotides specific for the disease causing entity.

An LNA-modified olignonucleotide contains one or more units of an LNA monomer, preferably one or more 2′-O, 4′-C-methylene bridge monomers (oxy-LNA). An LNA-modified oligonucleotide however also may contain other LNA units in addition to or in place of an oxy-LNA group. In particular, preferred additional LNA units include 2′-thio-LNA (thio-LNA), 2′-HN-LNA (amino-LNA), and 2′-N(R)-LNA (amino-R-LNA)) monomers in either the D-β or L-α configurations or combinations thereof. An LNA-modified oligonucleotide also may have other internucleoside linkages than the native phosphordiester, e.g. phosphoromonothioate, phosphorodithioate, and methylphosphonate linkages. The LNA-modified oligonucleotide can be fully modified with LNA (i.e. each nucleotide is an LNA unit), but it is generally preferred that the LNA-modified oligomers will contain other residues such as native DNA monomers, phosphoromonothioate monomers, methylphosphonate monomers or analogs thereof. In general, an LNA-modified oligonucleotide will contain at least about 5, 10, 15 or 20 percent LNA units, based on total nucleotides of the oligonucleotide, more typically at least about 20, 25, 30, 40, 50, 60, 70, 80 or 90 percent LNA units, based on total bases of the oligonucleotide.

An LNA-modified oligonucleotide used in accordance with the invention suitably is at least a 5-mer, 6-mer, 7-mer, 8-mer, 9-mer or 10-mer oligonucleotide, that is, the oligonucleotide is an oligomer containing at least 5, 6, 7, 8, 9, or 10 nucleotide residues, more preferably at least about 11 or 12 nucleotides. The preferred maximum size of the oligonucleotide is about 40, 50 or 60 nucleotides, more preferably up to about 25 or 30 nucleotides, and most preferably about between 12 and 20 nucleotides. While oligonucleotides smaller than 10-mers or 12-mers may be utilized they are more likely to hybridise with non-targeted sequences (due to the statistical possibility of finding exact sequence matches by chance in the human genome of 3×10⁹ bp), and for this reason may be less specific. In addition, a single mismatch may destabilise the hybrid thereby impairing its therapeutic function. While oligonucleotides larger than 40-mers may be utilised, synthesis, and cellular uptake may become somewhat more troublesome. Although specialised vehicles or oligonucleotide carriers will improve cellular uptake of large oligomers. Moreover, partial matching of long sequences may lead to non-specific hybridisation, and non-specific effects.

While in principle oligonucleotides having a sequence complementary to any region of the target mRNA find utility in the present invention, preferred are oligonucleotides capable of forming a stable duplex with a portion of the transcript lying within about 50 nucleotides (preferably within about 40 nucleotides) upstream (the 5′ direction), or about 50 (preferably 40) nucleotides downstream (the 3′ direction) from the translation initiation codon of the target mRNA. Also preferred are oligonucleotides which are capable of forming a stable duplex with a portion of the target mRNA transcript including the translation initiation codon.

LNA-modified oligonucleotides are useful for a number of therapeutic applications as indicated above. In general, therapeutic methods of the invention include administration of a therapeutically effective amount of an LNA-modified oligonucleotide to a mammal, particularly a human.

In antisense therapies, administered LNA-modified oligonucleotide contacts (interacts) with the targeted gene or RNA from the gene, whereby expression of the gene is modulated, and frequently expression is inhibited rather than increased. Such modulation of expression suitably will be at least a 10% or 20% difference relative to a control, more preferably at least a 30%, 40%, 50%, 60%, 70%, 80%, or 90% difference in expression relative to a control. It will be particularly preferred where interaction or contact with an LNA-modified oligonucleotide results in complete or essentially complete modulation of expression relative to a control, e.g. at least about a 95%, 97%, 98%, 99% or 100% inhibition of or increase in expression relative to control. A control sample for determination of such modulation can be comparable cells (in vitro or in vivo) that have not been contacted with the LNA-modified oligonucleotide.

The methods of the invention is preferably employed for treatment or prophylaxis of undesired cell growth (cancer), particularly for treatment of solid tumors as may occur in tissue such as lung, liver, prostate, brain, testes, stomach, intestine, bowel, or ovaries of a subject. The methods of the invention also may be employed to treat disseminated cancerous conditions such as leukemia. The methods of the invention are also preferably employed for treatment of diseases and disorders associated with inflammation, such as arthritic conditions, osteroarthritis, multiple sclerosis, and other autoimmune conditions, as well as various allergic conditions.

Definitions

As used herein, the term “proto-oncogene” refers to a normal, cellular human gene, the alteration of which gives rise to a transforming allele or “oncogene”.

As used herein, the term “oncogene” refers to a human gene that normally play a role in the growth of cells but, when overexpressed or mutated, can foster the growth of cancer.

As used herein, the term “DNA repair gene” refers to a gene that is part of a DNA repair pathway; that when altered, permits mutations to occur in the DNA of the organism.

As used herein, the term “infectious agent” refers to an organism which growth/multiplication leads to pathogenic events in the human body. Examples of such agents are: bacteria, fungi, protozoa, viruses, and parasites.

As used herein, the term “antisense oligonucleotide specific for” refers to an oligonucleotide having a sequence (i) capable of forming a stable complex with a portion of the targeted gene, e.g. by either strand invasion or triplex formation or (ii) capable of forming a stable duplex with a portion of a mRNA transcript of the targeted gene.

As used herein, the term “oligonucleotide” includes linear or circular oligomers of natural and/or modified monomers or linkages, including deoxyribonucleosides, ribonucleosides, substituted and alpha-anomeric forms thereof, polyamide nucleic acids (PNA), and the like, capable of specifically binding to a target polynucleotide by way of a regular pattern of monomer-to-monomer interactions, such as Watson-Crick type of base pairing, Hoogsteen or reverse Hoogsteen types of base pairing, or the like. The oligonucleotide may be composed of a single segment or may be composed of several segments. The oligonucleotide may be “chimeric”, i.e. composed of different segments, e.g. a DNA segment, a RNA segment, a PNA segment. The segment is in most cases composed of several consecutive monomers, but a segment can be as little as one residue. Segments may be linked in “register”, i.e. when the monomers are linked consecutively as in native DNA or linked via spacers. The spacers are intended to constitute a covalent “bridge” between the segments and have in preferred cases a length not exceeding 100 carbon atoms. The spacers may carry different functionalities, e.g. being charged, carry special nucleic acid binding properties (intercalators, groove binders, toxins, fluorophors etc.), being lipophilic, inducing special secondary structures like alanine containing peptides inducing alpha-helixes.

As used herein, the term “monomers” typically indicates monomers linked by phosphodiester bonds or analogs thereof to form oligonucleotides ranging in size from a few monomeric units, e.g., 3-4, to several hundreds of monomeric units. Analogs of phosphodiester linkages include: phosphorothioate, phosphorodithioate, methylphosphornates, phosphoroselenoate, phosphoramidate, and the like, as more fully described below. As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g., described generally by Scheit, Nucleotide Analogs, John Wiley, New York, 1980; and Freier & Altmann, Nucl. Acid. Res., 1997, 25(22), 4429-4443. Such analogs include synthetic nucleosides designed to enhance binding properties, e.g., duplex or triplex stability, specificity, or the like.

As used herein, the term “LNA-modified oligonucleotide” includes to any oligonucleotide either fully or partially modified with LNA monomers. Thus, an LNA-modified oligonucleotide may be composed entirely by LNA monomers, or a LNA-modified oligonucleotide may comprise one LNA monomer.

As used herein, the term “LNA monomer” typically refers to a nucleoside having a 2′-4′ cyclic linkage, as described in the International Patent Application WO 99/14226 and subsequent applications DK PA 1999 00381, U.S. provisional No. 60/127,357 and DK PA 1999 00603, U.S. provisional No. 60/133,273, all incorporated herein by reference. Preferred LNA monomers structures are exemplified in the formulae Ia and Ib below. In formula Ia the configuration of the furanose is denoted D-β, and in formula Ib the configuration is denoted L-α. Configurations which are composed of mixtures of the two, e.g. D-α and L-β, are also included.

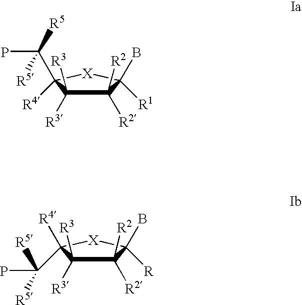

In Ia and Ib, X is oxygen, sulfur and carbon; B is a nucleobase, e.g. adenine, cytosine, 5-methylcytosine, isocytosine, pseudoisocytosine, guanine, thymine, uracil, 5-bromouracil, 5-propynyluracil, 5-propyny-6-fluoroluracil, 5-methylthiazoleuracil, 6-aminopurine, 2-aminopurine, inosine, diaminopurine, 7-propyne-7-deazaadenine, 7-propyne-7-deazaguanine. R¹, R² or R^{2′}, R³ or R^{3′}, R⁵ and R^{5′} are hydrogen, methyl, ethyl, propyl, propynyl, aminoalkyl, methoxy, propoxy, methoxy-ethoxy, fluoro, chloro. P designates the radical position for an internucleoside linkage to a succeeding monomer, or a 5′-terminal group, R³ or R^{3′} is an internucleoside linkage to a preceding monomer, or a 3′-terminal group. The internucleotide linkage may be a phosphate, phosphorthioate, phosphordithioate, phosphoramidate, phosphoroselenoate, phosphorodiselenoate, alkylphosphotriester, methyl phosphomates. The internucleotide linkage may also contain non-phosphorous linkers, hydroxylamine derivatives (e.g. —CH₂—NCH₃—O—CH₂—), hydrazine derivatives, e.g. —CH₂—NCH₃—NCH₃—CH₂, amid derivatives, e.g. —CH₂—CO—NH—CH₂—, CH₂—NH—CO—CH₂—. In Ia, R^{4′} and R^{2′} together designate —CH₂—O—, —CH₂—S—, —CH₂—NH— or —CH₂—NMe— where the oxygen, sulphur or nitrogen, respectively, is attached to the 2′-position. In Formula Ib, R^{4′} and R² together designate —CH₂—O—, —CH₂—S—, —CH₂—NH— or —CH₂—NMe— where the oxygen, sulphur or nitrogen, respectively, is attached to the 2-position (R² configuration).

Most preferred LNA monomer structures are structures in which X is oxygen (Formulae Ia, Ib); B is adenine, cytosine, 5-methylcytosine, isocytosine, pseudoisocytosine, guanine, thymine, uracil, 5-bromouracil, 5-propynyluracil, 5-propynyl-6-fluoroluracil, 6-aminopurine, 2-aminopurine, inosine, 2,6-diaminopurine, 7-propynyl-7-deazaadenine, 7-propynyl-7-deazaguanine; R¹, R² or R^{2′}, R³ or R^{3′}, R⁵ and R^{5′} are hydrogen; P is a phosphate, phosphorthioate, phosphordithioate, phosphoramidate, and methyl phosphomates; R³ or R^{3′} is an internucleoside linkage to a preceding monomer, or a 3′-terminal group. In Formula Ia, R^{4′} and R^{2′} together designate —CH₂—O—, —CH₂—S—, —CH₂—NH— or —CH₂—NMe— where the oxygen, sulphur or nitrogen, respectively, is attached to the 2′-position, and in Formula Ib, R^{4′} and R² together designate —CH₂—O—, —CH₂—S—, —CH₂—NH— or —CH₂—NMe— where the oxygen, sulphur or nitrogen, respectively, is attached to the 2′-position in the R² configuration.

As used herein, the term “corresponding unmodified reference oligonucleotide” refers to an oligonucleotide solely consisting of naturally occurring nucleotides that represent the same nucleobase sequence in the same orientation as the modified oligonucleotide.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, antisense oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the antisense oligonucleotide and the target nucleic acid of 1.5 μM. Thus, when used under physiological conditions, duplex or triplex formation will be substantially favoured over the state in which the antisense oligonucleotide and its target are dissociated. It is understood that a stable duplex or triplex may in some embodiments include mismatches between base pairs and/or among base triplets in the case of triplexes. Preferably, LNA modified antisense oligonucleotides of the invention form perfectly matched duplexes and/or triplexes with their target nucleic acids.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

As used herein, the term mRNA means the presently known mRNA transcript(s) of a targeted gene, and any further transcripts which may be elucidated.

### BRIEF DESCRIPTION OF THE DRAWINGS

FIG. 1 depicts results of nuclease assays on LNA containing DNA oligomers.

FIG. 2 depicts results of nuclease assays on control DNA oligomers.

FIG. 3 depicts RT-PCR results of FcεRIα mRNA from male Wistar rats.

### DETAILED DESCRIPTION OF THE INVENTION

According to preferred present invention, an LNA modified antisense oligonucleotide is designed to be specific for a gene which either causes, participates in or aggravates a disease state. This can be achieved by i) reducing or inhibiting the expression of the involved gene(s) or by ii) inducing or increasing the expression of a normally lowly expressed or unexpressed gene(s) the expression of which may mitigate or cure the disease state. Such induction or increases in the expression of a target gene may be achieved by for instance directing the antisense oligonucleotide against the mRNA of a gene that encodes a natural repressor of the target gene, by designing the antisense oligonucleotide in such a way that binding to its complementary sequence in the target mRNA will lead to an increase in target mRNA half-life and expression, or by using an oligonucleotide that can strand invade dsDNA to form a complex that can function as an initiation point for transcription of a downstream gene as described in Møllegaard et al. Proc. Natl. Acad. Sci. USA, 1994, 91(9), 3892-3895.

The utility of an LNA-modified oligonucleotide for modulation (including inhibition) of expression of a targeted gene can be readily determined by simple testing. Thus, as discussed above, an in vitro or in vivo expression system containing the targeted gene can be contacted with a particular LNA-modified oligonucleotide and levels of expression compared to control (same expression system that was not contacted with the LNA-modified oligonucleotide).

The LNA modified antisense oligonucleotide (vide infra) is administered to a patient by any of the routes described hereinafter.

Genes in Cancer

Cancer is a disease of genes gone awry. Genes that control the orderly replication of cells become damaged, allowing the cell to reproduce without restraint and eventually to spread into neighboring tissues and set up growths throughout the body. Cancer usually arises in a single cell. The cell's progress from normal to malignant to metastatic appears to follow a series of distinct steps, each one controlled by a different gene or set of genes. Several types of genes have been implicated. Oncogenes normally encourage cell growth; when mutated or overexpressed, they can flood cells with signals to keep on dividing. Tumor-suppressor genes normally restrain cell growth; when missing or inactivated by a mutation, they allow cells to grow and divide uncontrollably. DNA repair genes appear to trigger cancer—and perhaps other inherited disorders—not by spurring cell growth but by failing to correct mistakes that occur as DNA copies itself, enabling mutations accumulate at potentially thousands of sites.

The LNA modified antisense oligonucleotide may comprise antisense oligonucleotides specific to any tumour suppressor genes such as TP53, RB1, P16, oncogenes such as RAS and MYC or DNA repair genes such as MSH2 and MLH1 involved in the establishment and growth of a tumour. It may also be targeted against genes which are involved in tumour angiogenesis and metastasis such as for example the genes MMP-1 and MMP-2 which belong to the MMP family of matrix metalloproteinases that degrade connective tissue. Also, The LNA modified oligonucleotides may be directed against genes encoding multidrug transporter proteins such as the genes MDR-1 and MDR-2. Overexpression of such genes leads to multidrug resistance which is a major limitation to the success of current chemotherapy. Also, the LNA modified oligonucleotide may be directed against genes involved in the signal transduction pathway regulating cell growth such as cyclins and cyclin dependent kinases.

Table 1 below lists a number of genes involved in the establishment, growth, invasion and metastasis of tumors and genes involved in the development of resistance to chemotherapeutic drugs that are particularly interesting as antisense targets. It should be understood that many of the genes listed in table 1 are representatives of a larger gene family the other members of which also constitute potentially important antisense targets, e.g. ADAMTS-1 is a member of the ADAMs gene family that encode cellular disintegrins and metalloproteinases, MMP-1 is a member of the matrix metalloproteinases (MMPs) gene family that encode zinc-dependent endoproteinases, etc.

TABLE 1

| ABL1 | COT | GLI3 | PAI2 |
|---|---|---|---|
| ABL2 | CREB1 | GRO1 | PCNA |
| ABR | CREBBP | GRO2 | PDGFA |
| ADAM11 | CRK | GRO3 | PDGFB |
| ADAMTS-1 | CRKL | HCK | PDGFRA |
| AKT1 | CSF1 | HGF | PDGFRB |
| AKT2 | CSF1R | HKR3 | PIM1 |
| APC | CSF2 | HOX11 | PLAT |
| ARAF1 | CSF2RA | HOXA10 | PLAU |
| ARAF2 | CSF2RB | HOXB2 | PLAUR |
| AREG | CSF2RY | HPC1 | PLG |
| ARHA | CSF3R | HSPA9 | PMS1 |
| ARHB | D10S170 | HRAS | PMS2 |
| ARHC | DAP | IFNB1 | PPARA |
| AT | DAP3 | IFNG | PPARBP |
| AXL | DAPK1 | IFNGR1 | PPARG |
| BAD | DBCCR1 | IFNGR2 | PTCH |
| BAG1 | DCC | IRF4 | PVT1 |
| BAI1 | DDX6 | JUN | RAF1 |
| BAK1 | E2F1 | JUNB | RALA |
| BAP1 | E2F4 | JUND | RALB |
| BARD1 | E4F1 | KAI1 | RARA |
| BAX | EGF | KIT | RARB |
| BCL2 | EGFR | KRAS2 | RARG |
| BCL2A1 | EIF3S2 | LCK | RASA1 |
| BCL3 | EIF3S6 | LCN1 | RB1 |
| BCL5 | EIF4E | LCN2 | RBBP6 |
| BCL6 | EIFE4EBP1 | LCO | REL |
| BCNS | ELE1 | LCP1 | RELA |
| BCR | ELK1 | LCP2 | REQ |
| BCS | ELK3 | LPSA | RET |
| BL | ELK4 | LTA | RMYC |
| BLYM | EMP1 | LTB | ROS1 |
| BMI1 | EMS1 | LTK | RRAS |
| BMYC | EPHA1 | LYN | SEA |
| BRAF | EPHA3 | MAD | SET |
| BRCA1 | ERBAL2 | MADH4 | SIS |
| BRCA2 | ERBB2 | MAF | SKI |
| BRCD1 | ERBB3 | MAFG | SKIL |
| CALCR | ERBB4 | MAFK | SMARCB1 |
| CASP1 | ERG | MAP2K1 | SPI1 |
| CASP2 | ERPL1 | MAP2K4 | SPINK1 |
| CASP3 | ESR1 | MAP2K6 | SRC |
| CASP4 | ESR2 | MAP3K7 | ST5 |
| CASP5 | ESRRA | MAP3K8 | SUPT3H |
| CASP6 | ESRRB | MAP3K14 | SUPT5H |
| CASP13 | ESRRG | MAPKAPK3 | SUPT6H |
| CBL | ETS1 | M1S1 | TAF2A |
| CCNA1 | ETS2 | M4S1 | TAF2H |
| CCNA2 | ETV3 | M6P2 | TAL1 |
| CCNB1 | ETV4 | MPL | TF |
| CCNB2 | ETV6 | MAS1 | THPO |
| CCNC | EVI1 | MAX | THRA |
| CCND1 | EWSR1 | MCC | THRB |
| CCNP2 | FAT | MCF2 | TIAM1 |
| CCND3 | FER | MDM2 | TIM |
| CCNE1 | FES | MDR-1 | TIMP-1 |
| CCNE2 | FGD1 | MDR-2 | TIMP-2 |
| CCNF | FGF1 | MEL | TM4SF1 |
| CCNG1 | FGF2 | MEN1 | TNF |
| CCNG2 | FGF3 | MET | TP53 |
| CCNH | FGF4 | MGR-2 | TP53BP2 |
| CCNK | FGF5 | MLH1 | TP73 |
| CCNT1 | FGF6 | MMP-1 | VAV1 |
| CCNT2 | FGF7 | MMP-2 | VAV2 |
| CDC23 | FGF8 | MMP-3 | VDR |
| CDC25A | FGF9 | MMP-9 | VEGF |
| CDC25C | FGF10 | MNAT1 | VGF |
| CDC2L1 | FGF11 | MOS | VHL |
| CDC2L2 | FGF12 | MPL | WNT1 |
| CDC34 | FGF13 | MSH2 | WNT2 |
| CDH1 | FGF14 | MYB | WNT5A |
| CDH5 | FGF16 | MYBL1 | WT1 |
| CDH7 | FGF17 | MYBL2 | YES1 |
| CDK2 | FGF18 | MYC | |
| CDK3 | FGF19 | MYCL1 | |
| CDK4 | FGFR1 | MYCN | |
| CDK5 | FGFR2 | NBL1 | |
| CDK6 | FGFR3 | NF1 | |
| CDK7 | FGFR4 | NF2 | |
| CDK8 | FGR | NFKB2 | |
| CDK9 | FKHL1 | NKTR | |
| CDK10 | FLI1 | NOS2A | |
| CDKL1 | FLT1 | NOS2B | |
| CDKL2 | FMS | NOS2C | |
| CDKN1A | FPS | NOS3 | |
| CDKN1B | FOS | NOTCH4 | |
| CDKN1C | FOSB | NOV | |
| CDKN2A | FOSL1 | NRAS | |
| CDKN2B | FOSL2 | NRG1 | |
| CDKN2C | FYN | NRG2 | |
| CDKN2D | GADD45A | NTRK1 | |
| CDKN3 | GAK | ODC1 | |
| CDL4 | GLI | PACE | |
| CHES1 | GLI2 | PAI1 | |

It should be appreciated that in the above table 1, an indicated gene means the gene and all currently known variants thereof, including the different mRNA transcripts that the gene and its variants can give rise to, and any further gene variants which may be elucidated. In general, however, such variants will have significant homology (sequence identity) to a sequence of table 1 above, e.g. a variant will have at least about 70 percent homology (sequence identity) to a sequence of the above table 1, more typically at least about 75, 80, 85, 90, 95, 97, 98 or 99 homology (sequence identity) to a sequence of the above table 1. Homology of a variant can be determined by any of a number of standard techniques such as a BLAST program.

Sequences for the genes listed in Table 1 can be found in GenBank (http://www.ncbi.nlm.nih.gov/). The gene sequences may be genomic, cDNA or mRNA sequences. Preferred sequences are mammal genes containing the complete coding region and 5′ untranslated sequences. Particularly preferred are human cDNA sequences.

LNA modified antisense oligonucleotides may be used in combinations. For instance, a cocktail of several different LNA modified oligonucleotides, directed against different regions of the same gene, may be administered simultaneously or separately. In many cases, several cancer-promoting genes have to add up before a person will develop a malignant growth. In these cases combinations of LNA modified antisense oligonucleotides specific for the different genes may be administered simultaneously or separately. LNA modified oligonucleotides may also be administered in combination with standard chemotherapeutic drugs. For instance, an LNA modified oligonucleotide directed against a multidrug transporter gene such as MDR-1, MDR-2 or MGR-2, or a combination of LNA modified oligonucleotides directed against two or more of such genes, may be used in combination with standard chemotherapeutic drugs in patients displaying the multidrug resistance phenotype.

Genes in Inflammatory/Allergic Diseases

Inflammatory diseases can afflict every major organ system. Inflammation has evolved as a defense mechanism that gets rid of or prevents the spread of substances foreign to the human body. But many times, the function of molecular components in this normally efficient system can go awry. Common examples of inflammatory diseases are asthma, lupus, multiple sclerosis, osteoarthritis, psoriasis, Crohn's disease and rheumatoid arthritis.

According to the invention LNA modified oligonucleotides may be used to modulate the expresssion of genes involved in inflammatory diseases. Tables 2 through 5 lists a number of such genes that are particularly interesting as antisense targets; table 2 (CD markers), table 3 (adhesion molecules) table 4 (chemokines and chemokine receptors), and table 5 (interleukins and their receptors). Also included as particularly interesting antisense targets are the genes encoding the immunoglubulin E (IgE) and the IgE-recptor (FcεRIα) as well as the genes for the other immunoglubulins, IgG(1-4), IgA1, IgA2, IgM, IgE, and IgD encoding free and membrane bound immunoglobulin's and the genes encoding their corresponding receptors.

TABLE 2
CD markers

| | | | | |
|---|---|---|---|---|
| CD1a-d | CD30 | CD61 | CD91 | CD121 |
| CD2 | CD31 | CD62E | CDw92 | CD122 |
| CD3 | CD32 | CD62L | CD93 | CDw123 |
| CD4 | CD33 | CD62P | CD94 | CD124 |
| CD5 | CD34 | CD63 | CD95 | CDw125 |
| CD6 | CD35 | CD64 | CD96 | CD126 |
| CD7 | CD36 | CD65 | CD97 | CD127 |
| CD8 | CD37 | CD66a-e | CD98 | CDw128 |
| CD9 | CD38 | CD67 | CD99 | CD129 |
| CD10 | CD39 | CD68 | CD100 | CD130 |
| CD11a | CD40 | CD69 | CD101 | CDw131 |
| CD11b | CD41 | CD70 | CD102 | CD132 |
| CD11c | CD42a-d | CD71 | CD103 | CD133 |
| CDw12 | CD43 | CD72 | CD104 | CD134 |
| CD13 | CD44 | CD73 | CD105 | |
| CD14 | CD45 | CD74 | CD106 | |
| CD15 | CD46 | CDw75 | CD107a,b | |
| CD16 | CD47 | CDw76 | CDw08 | |
| CDw17 | CD48 | CD77 | CD109 | |
| CD18 | CD49a-f | CDw78 | CD110 | |
| CD19 | CD50 | CD79a,b | CD111 | |
| CD20 | CD51 | CD80 | CD112 | |
| CD21 | CD52 | CD81 | CD113 | |
| CD22 | CD53 | CD82 | CD114 | |
| CD23 | CD54 | CD83 | CD115 | |
| CD24 | CD55 | CDw84 | CD116 | |
| CD25 | CD56 | CD85 | CD117 | |
| CD26 | CD57 | CD86 | CD118 | |
| CD27 | CD58 | CD87 | CD119 | |
| CD28 | CD59 | CD88 | CD120a,b | |
| CD29 | CDw60 | CD89 | | |
| CD30 | | CD90 | | |

TABLE 3
Adhesion molecules

| | | | | |
|---|---|---|---|---|
| L-selectin | TCRγ/δ | BB-1 | Integrin α7 | Integrin α6 |
| P-selectin | CD28 | N-cadherin | Integrin α8 | Integrin β5 |
| E-selectin | LFA-3 | E-cadherin P- | IntegrinαV | Integrin αV |
| HNK-1 | PECAM-1 | cadherin | Integrin β2 | Integrin β6 |
| Sialyl- | VCAM-1 | Integrin β1 | Integrin αL | Integrin αV |
| Lewis X | | | | |
| CD15 | ICAM-2 | Integrin α1 | Integrin αM | Integrin β7 |
| LFA-2 | ICAM-3 | Integrin α2 | IntegrinαX | IntegrinαIEL |
| CD22 | Leukosialin | Integrin α3 | Integrin β3 | Integrin α4 |
| ICAM-1 | HCAM | Integrin α4 | IntegrinαV | Integrin β8 |
| N-CAM | CD45RO | Integrin α5 | IntegrinαIib | Integrin αV |
| Ng-CAM | CD5 | Integrin α6 | Integrin β4 | |
| TCRα/β | HPCA-2 | | | |

TABLE 4
Chemokines and Chemokine receptors

| C-X-C | | | | hemokine |
|---|---|---|---|---|
| chemokines | C-C chemokines | | C chemokines | eceptors |
| IL-8 | MCAF/MCP-1 | ABCD-1 | Lymphotactin | CCR1 |
| NAP-2 | MIP-1 α,β | LMC | | CCR2 |
| GRO/MGSA | RANTES | AMAC-1 | | CCR3 |
| γ IP-10 | I-309 | NCC-4 | | CCR4 |
| ENA-78 | CCF18 | LKN-1 | | CCR5 |
| SDF-1 | SLC | STCP-1 | | CCR6 |
| I-TAC | TARC | TECK | | CCR7 |
| LIX | PARC | EST | | CCR8 |
| SCYB9 | LARC | MDC | | CXCR1 |
| B cell- | EBI 1 | Eotaxin | | CXCR2 |
| attracting | | | | |
| chemokine 1 | HCC-1 | | | CXCR3 |
| | HCC-4 | | | CXCR4 |
| | | | | CXCR5 |
| | | | | CX₃CR |

TABLE 5
Interleukins and their receptors

| | | | | |
|---|---|---|---|---|
| G-CSF | IL-2 Rα | IL-8 | IL-16 | TGF-β1 |
| G-CSF R | IL-2 Rβ | IL-9 | IL-17 | TGF-β1,2 |
| GM-CSF | IL-2 Rγ | IL-9 R | IL-18 | TGF-β2 |
| IFN-γ | IL-3 | IL-10 | PDGF | TGF-β3 |
| IGF-I | IL-3 Rα | IL-10 R | PDGF A Chain | TGF-β5 |
| IGF-I R | IL-4 | IL-11 | PDGF-AA | LAP TGF-β1 |
| IGF-II | IL-4 R | IL-11 R | PDGF-AB | Latent TGF-β1 |
| IL-1α | IL-5 | IL-12 | PDGF B Chain | TGF-β bpl |
| IL-1β | IL-5 Rα | IL-12 p40 | PDGF-BB | TGF-β RII |
| IL-1 RI | IL-6 | IL-12 p70 | PDGF Rα | TGF-β RIII |
| IL-1 RII | IL-6 R | IL-13 | PDGF Rβ | |
| IL-1rα | IL-7 | IL-13 Rα | TGF-α | |
| IL-2 | IL-7 R | IL-15 | TGF-β | |

It should be appreciated that in the above tables 2 through 5, an indicated gene means the gene and all currently known variants thereof, including the different mRNA transcripts that the gene and its variants can give rise to, and any further gene variants which may be elucidated. In general, however, such variants will have significant homology (sequence identity) to a sequence of a table above, e.g. a variant will have at least about 70 percent homology (sequence identity) to a sequence of the above tables 2-5, more typically at least about 75, 80, 85, 90, 95, 97, 98 or 99 homology (sequence identity) to a sequence of the above tables 2-5. Homology of a variant can be determined by any of a number of standard techniques such as a BLAST program.

Sequences for the genes listed in Tables 2-5 can be found in GenBank (http://www.ncbi.nlm.nih.gov/). The gene sequences may be genomic, cDNA or mRNA sequences. Preferred sequences are mammal genes containing the complete coding region and 5′ untranslated sequences. Particularly preferred are human cDNA sequences.

As in the case of LNA modified antisense oligonucleotides against cancer, LNA modified oligonucleotides against genes involved in inflammatory/allergic diseases may be used in combinations. For instance, a cocktail of several different LNA modified oligonucleotides, directed against different regions of the same gene, may be administered simultaneously or separately. Also, combinations of LNA modified antisense oligonucleotides specific for different genes, such as for instance the IgE gene and the IgE-recptor (FcεRIα), may be administered simultaneously or separately. LNA modified oligonucleotides may also be administered in combination with other antiinflammatory drugs.

In the practice of the present invention, target genes may be single-stranded or double-stranded DNA or RNA; however, single-stranded DNA or RNA targets are preferred. It is understood that the target to which the antisense oligonucleotides of the invention are directed include allelic forms of the targeted gene and the corresponding mRNAs including splice variants. There is substantial guidance in the literature for selecting particular sequences for antisense oligonucleotides given a knowledge of the sequence of the target polynucleotide, e.g., Peyman and Ulmann, Chemical Reviews, 90:543-584, 1990; Crooke, Ann. Rev. Pharmacal. Toxicol., 32:329-376 (1992); and Zamecnik and Stephenson, Proc. Natl. Acad. Sci., 75:280-284 (1974). Preferably, the sequences of antisense compounds are selected such that the G-C content is at least 60%. Preferred mRNA targets include the 5′ cap site, tRNA primer binding site, the initiation codon site, the mRNA donor splice site, and the mRNA acceptor splice site, e.g., Goodchild et al., U.S. Pat. No. 4,806,463.

Where the target polynucleotide comprises a mRNA transcript, oligonucleotides complementary to and hybridizable with any portion of the transcript are, in principle, effective for inhibiting translation, and capable of inducing the effects herein described. It is believed that translation is most effectively inhibited by blocking the mRNA at a site at or near the initiation codon. Thus, oligonucleotides complementary to the 5′-region of mRNA transcript are preferred. Oligonucleotides complementary to the mRNA, including the initiation codon (the first codon at the 5′ end of the translated portion of the transcript), or codons adjacent to the initiation codon, are preferred.

While antisense oligomers complementary to the 5′-region of the mRNA transcripts are preferred, particularly the region including the initiation codon, it should be appreciated that useful antisense oligomers are not limited to those oligomers complementary to the sequences found in the translated portion of the mRNA transcript, but also includes oligomers complementary to nucleotide sequences contained in, or extending into, the 5′- and 3′-untranslated regions. Antisense oligonucleotides complementary to the 3′-untranslated region may be particularly useful in regard to increasing the half-life of a mRNA thereby potentially up-regulating its expression.

It is well known that many sequences in a mRNA cannot be addressed by standard oligonucleotides employing oligonucleotides of moderate affinity e.g. oligonucleotides composed of DNA and/or RNA monomers or the currently used analogues. It is believed that this problem is primarily due to intra-molecular base-pairings structures in the target mRNA. The use of appropriately designed LNA modified oligonucleotides can effectively compete with such structures due to the increased affinity of such oligonucleotides compared to the unmodified reference oligonucleotides. Thus, LNA can be used to design antisense oligonucleotides with a greater therapeutic potential than that of current antisense oligonucleotides.

LNA modified antisense oligonucleotides of the invention may comprise any polymeric compound capable of specifically binding to a target oligonucleotide by way of a regular pattern of monomer-to-nucleoside interactions, such as Watson-Crick type of base pairing, Hoogsteen or reverse Hoogsteen types of base pairing, or the like. An LNA modified antisense oligonucleotide will have higher affinity to the target sequence compared with the corresponding unmodified reference oligonucleotide of similar sequence.

A particular aspect of the invention is the use of LNA monomers to improve on the target specificity and cellular uptake and distribution of current oligonucleotides e.g. oligonucleotides consisting of standard DNA and/or RNA monomers and/or current DNA monomer analogues. This can be achieved by substituting some of the monomers in the current oligonucleotides by LNA monomers whilst at the same time reducing the size of the oligonucleotide to compensate for the increased affinity imposed by the incorporation of LNA monomers. Such short LNA-modified oligonucleotides exhibits as high or higher affinity than the unmodified oligonucleotide but better target specificity and enhanced cellular uptake and distribution because of the reduced size. It is preferred that such LNA-modified oligonucleotides contain less than 70%, more preferably less than 60%, most preferably less than 50% LNA monomers and that their sizes are between 10 and 25 nucleotides, more preferably between 12 and 20 nucleotides.

A further aspect of the invention is to use different LNA monomers in the oligonculeotide such as for example the oxy-LNA, thio-LNA or amino-LNA monomers. The use of such different monomers offers a means to “fine tune” the chemical, physical, biological and pharmacological properties of the oligonucleotide thereby facilitating improvement in their safety and efficacy profiles when used as antisense drugs.

LNA-modified compounds of the invention may also contain pendent groups or moieties, either as part of or separate from the basic repeat unit of the polymer, to enhance specificity, improve nuclease resistance, delivery, cellular uptake, cell and organ distribution, in-vivo transport and clearance or other properties related to efficacy and safety, e.g., cholesterol moieties, duplex intercalators such as acridine, poly-L-lysine, “end-capping” with one or more nuclease-resistant linkage groups such as phosphoromonothioate, and the like.

Many pendant groups or moieties, when attached to an oligo, decrease its affinity for its complementary target sequence. Because the efficacy of an antisense oligo depends to a significant extend on its ability to bind with high affinity to its target sequence, such pendant groups or moieties, even though being potentially useful, are not suitable for use with oligonucleotides composed of standard DNA, RNA or other moderate affinity analogues. Incorporation of LNA monomers into such oligonucleotides can be used as a means to compensate for the affinity loss associated with such pendant groups or moieties. Thus, LNA offers a general means for extracting the benefits of affinity decreasing pendant groups or moieties.

Incorporation of LNA monomers into a standard DNA or RNA oligonuclotide will increase its resistance towards nucleases (endonucleases and exonucleases), the extent of which will depend on the number of LNA monomers used and their position in the oligonucleotide. Nuclease resistance of LNA-modified oligonucleotides can be further enhanced by providing nuclease-resistant internucleosidic linkages. Many such linkages are known in the art, e.g., phosphorothioate: Zon and Geiser, Anti-Cancer Drug Design, 6:539-568 (1991); U.S. Pat. Nos. 5,151,510; 5,166,387; and 5,183,885; phosphorodithioates: Marshall et al., Science, 259:1564-1570 (1993); Caruthers and Nielsen, International Patent Application PCT/US89/02293; phosphoramidates, e.g., —O₂P(═O)(NR), where R may be hydrogen or C1-C3 alkyl; Jager et al., Biochemistry, 27:7237-7246 (1988); Froehler et al., International application PCT/US90/03138; peptide nucleic acids: Nielsen et al., Anti-Cancer Drug Design, 8:53-63 (1993), International application PCT/EP92/01220; methylphosphonates: U.S. Pat. Nos. 4,507,433; 4,469,863; and 4,757,055; and P-chiral linkages of various types, especially phosphorothioates, Stec et al., European patent application 506,242 (1992) and Lesnikowski, Bioorganic Chemistry, 21:127-155 (1993). Additional nuclease linkages include phosphoroselenoate, phosphorodiselenoate, alkylphosphotriester such as methyl- and ethylphosphotriester, carbonate such as carboxymethyl ester, carbamate, morpholino carbamate, 3′-thioformacetal, silyl such as dialkyl (C1-C6)- or diphenylsilyl, sulfamate ester, and the like. Such linkages and methods for introducing them into oligonucleotides are described in many references, e.g., reviewed generally by Peyman and Ulmann, Chemical Reviews 90:543-584 (1990); Milligan et al., J. Med. Chem., 36:1923-1937 (1993); Matteucci et al., International application PCT/US91/06855.

Resistance to nuclease digestion may also be achieved by modifying the internucleotide linkage at both the 5′ and 3′ termini with phosphoroamidites according to the procedure of Dagle et al., Nucl. Acids Res. 18, 4751-4757 (1990).

Preferably, phosphorus analogs of the phosphodiester linkage are employed in the compounds of the invention, such as phosphorothioate, phosphorodithioate, phosphoramidate, or methylphosphonate. More preferably, phosphoromonothioate is employed as the nuclease resistant linkage.

Phosphorothioate oligonucleotides contain a sulfur-for-oxygen substitution in the internucleotide phosphodiester bond. Phosphorothioate oligonucleotides combine the properties of effective hybridization for duplex formation with substantial nuclease resistance, while retaining the water solubility of a charged phosphate analogue. The charge is believed to confer the property of cellular uptake via a receptor (Loke et al., Proc. Natl. Acad. Sci., 86, 3474-3478 (1989)).

It is understood that in addition to the preferred linkage groups, compounds of the invention may comprise additional modifications, e.g., boronated bases, Spielvogel et al., U.S. Pat. No. 5,130,302; cholesterol moieties, Shea et al., Nucleic Acids Research, 18:3777-3783 (1990) or Letsinger et al., Proc. Natl. Acad. Sci., 86:6553-6556 (1989); and 5-propynyl modification of pyrimidines, Froehler et al., Tetrahedron Lett., 33:5307-5310 (1992).

Preferably, LNA-modified oligonucleotides compounds of the invention are synthesized according to the methods as described in International Patent Application WO 99/14226, which is fully incorporated herein by reference.

In embodiments where triplex formation is desired, there are constraints on the selection of target sequences. Generally, third strand association via Hoogsteen type of binding is most stable along homopyrimidine-homopurine tracks in a double stranded target. Usually, base triplets form in T-A*T or C-G*C motifs (where “-” indicates Watson-Crick pairing and “*” indicates Hoogsteen type of binding); however, other motifs are also possible. For example, Hoogsteen base pairing permits parallel and antiparallel orientations between the third strand (the Hoogsteen strand) and the purine-rich strand of the duplex to which the third strand binds, depending on conditions and the composition of the strands. There is extensive guidance in the literature for selecting appropriate sequences, orientation, conditions, nucleoside type (e.g., whether ribose or deoxyribose nucleosides are employed), base modifications (e.g., methylated cytosine, and the like) in order to maximize, or otherwise regulate, triplex stability as desired in particular embodiments, e.g., Roberts et al., Proc. Natl. Acad. Sci., 88:9397-9401 (1991); Roberts et al., Science, 58:1463-1466 (1992); Distefano et al., Proc. Natl. Acad. Sci., 90:1179-1183 (1993); Mergny et al., Biochemistry, 30:9791-9798 (1992); Cheng et al., J. Am. Chem. Soc., 114:4465-4474 (1992); Beal and Dervan, Nucleic Acids Research, 20:2773-2776 (1992); Beal and Dervan, J. Am. Chem. Soc., 114:4976-4982; Giovannangeli et al., Proc. Natl. Acad. Sci., 89:8631-8635 (1992); Moser and Dervan, Science, 238:645-650 (1987); McShan et al., J. Biol. Chem., 267:5712-5721(1992); Yoon et al., Proc. Natl. Acad. Sci., 89:3840-3844 (1992); and Blume et al., Nucleic Acids Research, 20:1777-1784 (1992).

The length of the oligonucleotide moieties is sufficiently large to ensure that specific binding will take place only at the desired target polynucleotide and not at other fortuitous sites, as explained in many references, e.g., Rosenberg et al., International application PCT/US92/05305; or Szostak et al., Meth. Enzymol, 68:419-429 (1979). The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, whether modifications to enhance binding or specificity are present, whether duplex or triplex binding is desired, and the like. Usually, antisense compounds of the invention have lengths in the range of about 12 to 40 nucleotides. More preferably 30 nucleotides; and most preferably, they have lengths in the range of about 12 to 20 nucleotides.

In general, the LNA-modified oligonucleotides used in the practice of the present invention will have a sequence which is completely complementary to a selected portion of the target polynucleotide. Absolute complementarity, however, is not required, particularly in larger oligomers. Thus, reference herein to an “LNA-modified oligonucleotide sequence complementary to” a target polynucleotide does not necessarily mean a sequence having 100% complementarity with the target segment. In general, any oligonucleotide having sufficient complementarity to form a stable duplex with the target (e.g. a gene or its mRNA transcript) that is, an oligonucleotide which is “hybridizable”, is suitable. Stable duplex formation depends on the sequence and length of the hybridizing oligonucleotide and the degree of complementarity with the target polynucleotide. Generally, the larger the hybridizing oligomer, the more mismatches may be tolerated. More than one mismatch will probably not be tolerated for antisense oligomers of less than about 11 nucleotides. One skilled in the art may readily determine the degree of mismatching which may be tolerated between any given antisense oligomer and the target sequence, based upon the melting point, and therefore the thermal stability, of the resulting duplex. In general, an LNA-modified oligonucleotide will be at least about 60% complementary to a selected portion of the target polynucleotide, more typically an LNA-modified oligonucleotide will be at least about 70, 80, 90 or 95 percent complementary to a selected portion of the target polynucleotide.

The ability of an LNA-modified oligonuleotide to hybridize to a target polynucleotide also can be readily determined empirically in vitro. In particular, preferred LNA-modified oligonucleotides will bind a target polynucleotide under the following moderately stringent conditions (referred to herein as “normal stringency” conditions): use of a hybridization buffer comprising 100 mM NaCl, 0.1 mM EDTA and 10 mM phosphate buffer, pH 7.0 at a temperature of 37° C. Particularly preferred LNA-modified oligonucleotides will bind a target polynucleotide under the following highly stringent conditions (referred to herein as “high stringency” conditions): use of a hybridization buffer comprising 0.1 mM EDTA and 10 mM phosphate buffer, pH 7.0 at a temperature of 42° C.

Preferably, the thermal stability of hybrids formed by the LNA-modified oligonucleotides of the invention are determined by way of melting, or strand dissociation, curves. The temperature of fifty percent strand dissociation is taken as the melting temperature, Tₘ, which, in turn, provides a convenient measure of stability. Tₘ measurements are typically carried out in a saline solution at neutral pH with target and LNA-modified oligonucleotide concentrations at between about 0.5-5 μM. Typical conditions are as follows: 100 mM NaCl and 0.1 mM EDTA in a 10 mM sodium phosphate buffer (pH 7.0) and 1.5 μM of each oligonucleotide. Data for melting curves are accumulated by heating a sample of the antisense oligonucleotide/target polynucleotide complex from room temperature to about 90° C. As the temperature of the sample increases, absorbance of 260 nm light is monitored at 1° C. intervals, e.g., using e.g. a Cary (Australia) model 1E or a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of LNA modified antisense oligonucleotides of different lengths and compositions.

Pharmaceutical compositions of the invention include a pharmaceutical carrier that may contain a variety of components that provide a variety of functions, including regulation of drug concentration, regulation of solubility, chemical stabilization, regulation of viscosity, absorption enhancement, regulation of pH, and the like. The pharmaceutical carrier may comprise a suitable liquid vehicle or excipient and an optional auxiliary additive or additives. The liquid vehicles and excipients are conventional and commercially available. Illustrative thereof are distilled water, physiological saline, aqueous solutions of dextrose, and the like. For water soluble formulations, the pharmaceutical composition preferably includes a buffer such as a phosphate buffer, or other organic acid salt, preferably at a pH in the range of 6.5 to 8. For formulations containing weakly soluble antisense compounds, micro-emulsions may be employed, for example by using a nonionic surfactant such as polysorbate 80 in an amount of 0.04-0.05% (w/v), to increase solubility. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences, e.g., Remington's Pharmaceutical Science, latest edition (Mack Publishing Company, Easton, Pa.).

LNA-modified oligonucleotides of the invention include the pharmaceutically acceptable salts thereof, including those of alkaline earth salts, e.g., sodium or magnesium, ammonium or NX₄⁺, wherein X is C₁-C₄ alkyl. Other pharmaceutically acceptable salts include organic carboxylic acids such as formic, acetic, lactic, tartaric, malic, isethionic, lactobionic, and succinic acids; organic sulfonic acids such as methanesulfonic, ethanesulfonic, tolouenesulfonic acid and benzenesulfonic; and inorganic acids such as hydrochloric, sulfuric, phosphoric, and sulfamic acids. Pharmaceutically acceptable salts of a compound having a hydroxyl group include the anion of such compound in with a suitable cation such as Na⁺, NH₄⁺, or the like.

LNA-modified oligonucleotides of the invention are preferably administered to a subject orally or topically but may also be administered intravenously by injection. The vehicle is designed accordingly. Alternatively, the oligonucleotide may be administered subcutaneously via controlled release dosage forms.

In addition to administration with conventional carriers, the antisense oligonucleotides may be administered by a variety of specialized oligonucleotide delivery techniques. Sustained release systems suitable for use with the pharmaceutical compositions of the invention include semi-permeable polymer matrices in the form of films, microcapsules, or the like, comprising polylactides; copolymers of L-glutamic acid and gamma-ethyl-L-glutamate, poly(2-hydroxyethyl methacrylate), and like materials, e.g., Rosenberg et al., International application PCT/US92/05305.

The oligonucleotides may be encapsulated in liposomes for therapeutic delivery, as described for example in Liposome Technology, Vol. II, Incorporation of Drugs, Proteins, and Genetic Material, CRC Press. The oligonucleotide, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, ionic surfactants such as diacetylphosphate, stearylamine, or phosphatidic acid, and/or other materials of a hydrophobic nature. Also comprized are the novel cationic amphiphiles, termed “molecular umbrellas, that are described in (DeLong et al, Nucl. Acid. Res., 1999, 27(16), 3334-3341).

The oligonucleotides may be conjugated to peptide carriers. Examples are poly(L-lysine) that significantly increased cell penetration and the antenepedia transport peptide. Such conjugates are described by Lemaitre et al., Proc. Natl. Acad. Sci. USA, 84, 648-652 (1987). The procedure requires that the 3′-terminal nucleotide be a ribonucleotide. The resulting aldehyde groups are then randomly coupled to the epsilon-amino groups of lysine residues of poly(L-lysine) by Schiff base formation, and then reduced with sodium cyanoborohydride. This procedure converts the 3′-terminal ribose ring into a morpholine structure antisense oligomers.

The peptide segment can also be synthesised by strategies which are compatible with DNA/RNA synthesis e.g. Mmt/Fmoc strategies. In that case the peptide can be synthesised directly before or after the oligonucleotide segment. Also methods exist to prepare the peptide oligonucleotide conjugate post synthetically, e.g. by formation of a disulfide bridge.

The LNA modified oligonucleotides may also be synthesized as pro-drugs carrying lipophilic groups, such as for example methyl-SATE (S-acetylthioethyl) or t-Bu-SATE (S-pivaloylthioethyl) protecting groups, that confers nuclease resistance to the oligo, improve cellular uptake and selectively deprotects after entry into the cell as described in Vives et al. Nucl. Acids Res. 1999, Vol. 27, 4071-4076. The LNA modified oligonucleotide may also be synthesized as circular molecules in which the 5′ and 3′ ends of the oligonucleotides are covalently linked or held together by an affinity pair one member of which is attached covalently to the 5′ end and the other attached covalently to the 3′ end. Such circularisation will protect the oligonucleotide against degradation by exonucleases and may also improve cellualr uptake and distribution. In one aspect of the invention the moity linking the 5′ and 3′ end of a circular oligonucleotide is cleaved automatically upon entry into any type of human or vertebrate cell thereby linearising the oligonucleotide and enabling it to efficiently hybridise to its target sequence. In another aspect, the moity linking the 5′ and 3′ ends of the oligonucleotide is so designed that cleavage preferably occurs only in the particular type of cells that expresses the mRNA that is the target for the antisense oligonucleotide. For instance, a circular antisense oligonucleotide directed against a gene involved in cancer may be brought into action by linearisation only in the subset of cells expressing the malignant gene. Likewise, circular antisense oligonucleotides directed against bacterial or viral genes may be activated in only infected cells.

LNA modified antisense compounds of the invention also include conjugates of such oligonucleotides with appropriate ligand-binding molecules. The oligonucleotides may be conjugated for therapeutic administration to ligand-binding molecules which recognize cell-surface molecules, such as according to International Patent Application WO 91/04753. The ligand-binding molecule may comprise, for example, an antibody against a cell surface antigen, an antibody against a cell surface receptor, a growth factor having a corresponding cell surface receptor, an antibody to such a growth factor, or an antibody which recognizes a complex of a growth factor and its receptor. Methods for conjugating ligand-binding molecules to oligonucleotides are detailed in WO 91/04753.

In particular, the growth factor to which the antisense oligonucleotide may be conjugated, may comprise transferrin or folate. Transferrin-polylysine-oligonucleotide complexes or folate-polylysine-oligonucleotide complexes may be prepared for uptake by cells expressing high levels of transferrin or folate receptor. The preparation of transferrin complexes as carriers of oligonucleotide uptake into cells is described by Wagner et al., Proc. Natl. Acad. Sci. USA 87, 3410-3414 (1990). Inhibition of leukemia cell proliferation by transferrin receptor-mediated uptake of c-myb antisense oligonucleotides conjugated to transferrin has been demonstrated by Citro et al., Proc. Natl. Acad. Sci. USA., 89, 7031-7035 (1992). Cellular delivery of folate-macromolecule conjugates via folate receptor endocytosis, including delivery of an antisense oligonucleotide, is described by Low et al., U.S. Pat. No. 5,108,921. Also see, Leamon et al., Proc. Natl. Acad. Sci. 88, 5572 (1991).

A preferred method of administration of oligonucleotides comprises either, topical, systemic or regional perfusion, as is appropriate. According to a method of regional perfusion, the afferent and efferent vessels supplying the extremity containing the lesion are isolated and connected to a low-flow perfusion pump in continuity with an oxygenator and a heat exchanger. The iliac vessels may be used for perfusion of the lower extremity. The axillary vessels are cannulated high in the axilla for upper extremity lesions. Oligonucleotide is added to the perfusion circuit, and the perfusion is continued for an appropriate time period, e.g., one hour. Perfusion rates of from 100 to 150 ml/minute may be employed for lower extremity lesions, while half that rate should be employed for upper extremity lesions. Systemic heparinization may be used throughout the perfusion, and reversed after the perfusion is complete. This isolation perfusion technique permits administration of higher doses of chemotherapeutic agent than would otherwise be tolerated upon infusion into the arterial or venous systemic circulation.

For systemic infusion, the oligonucleotides are preferably delivered via a central venous catheter, which is connected to an appropriate continuous infusion device. Indwelling catheters provide long term access to the intravenous circulation for frequent administration of drugs over extended time periods. They are generally surgically inserted into the external cephalic or internal jugular vein under general or local anesthesia. The subclavian vein is another common site of catheterization. The infuser pump may be external, or may form part of an entirely implantable central venous system such as the INFUSAPORT system available from Infusaid Corp., Norwood, Mass. and the PORT-A-CATH system available from Pharmacia Laboratories, Piscataway, N.J. These devices are implanted into a subcutaneous pocket under local anesthesia. A catheter, connected to the pump injection port, is threaded through the subclavian vein to the superior vena cava. The implant contains a supply of oligonucleotide in a reservoir which may be replenished as needed by injection of additional drug from a hypodermic needle through a self-sealing diaphragm in the reservoir. Completely implantable infusers are preferred, as they are generally well accepted by patients because of the convenience, ease of maintenance and cosmetic advantage of such devices.

LNA-modified oligonucleotides of the invention may be introduced by any of the methods described in U.S. Pat. No. 4,740,463, incorporated herein by reference. One technique is transfection; which can be done by several different methods. One method of transfection involves the addition of DEAE-dextran to increase the uptake of the naked DNA molecules by a recipient cell. See McCutchin, J. H. and Pagano, J. S., J. Natl. Cancer Inst. 41, 351-7 (1968). Another method of transfection is the calcium phosphate precipitation technique which depends upon the addition of Ca<++> to a phosphate-containing DNA solution. The resulting precipitate apparently includes DNA in association with calcium phosphate crystals. These crystals settle onto a cell monolayer; the resulting apposition of crystals and cell surface appears to lead to uptake of the DNA. A small proportion of the DNA taken up becomes expressed in a transfectant, as well as in its clonal descendants. See Graham, F. L. and van der Eb, A. J., Virology 52, 456-467 (1973) and Virology 54, 536-539 (1973).

Transfection may also be carried out by cationic phospholipid-mediated delivery. In particular, polycationic liposomes can be formed from N-[1-(2,3-di-oleyloxy)propyl]-N,N,N-trimethylammonium chloride (DOT-MA). See Felgner et al., Proc. Natl. Acad. Sci., 84, 7413-7417 (1987) (DNA-transfection); Malone et al., Proc. Natl. Acad. Sci., 86, 6077-6081 (1989) (RNA-transfection).

Particulate systems and polymers for in vitro and in vivo delivery of polynucleotides were extensively reviewed by Felgner in Advanced Drug Delivery Reviews 5, 163-187 (1990). Techniques for direct delivery of purified genes in vivo has been reviewed by Felgner in Nature 349, 351-352 (1991). Such methods of direct delivery of polynucleotides may be utilized for local delivery of either exogenous antisense oligonucleotides.

The LNA modified antisense oligonucleotides may be used as the primary therapeutic for the treatment of the disease state, or may be used in combination with non-oligonucleotide drugs. One example of this is in Multi Drug Resistance (MDR) in which the tumour cells acquire resistance to chemotherapeutic agents. This resistance is a result of over expression of particular genes such as for instance the MDR-1 and MDR-2 genes. An antisense oligonucleotide can reduce or inhibit the expression of the genes, and thereby “reinstall” responsiveness to chemotherapeutic drugs of the otherwise resistant tumour cells. Typical examples of chemotherapeutic agents that could be used in combination with antisense oligonucleotide drugs include drugs such as dacarbazine, mitoxantrone, cyclophosphamide, docetaxel, VP-16, cis-platinum, actinomycin D, doxorubicin, taxol and methotrexate.

For systemic or regional in vivo administration, the amount of LNA-modified oligonucleotides may vary depending on the nature and extent of the disease, the particular oligonucleotides utilized, and other factors. The actual dosage administered may take into account the size and weight of the patient, whether the nature of the treatment is prophylactic or therapeutic in nature, the age, health and sex of the patient, the route of administration, whether the treatment is regional or systemic, and other factors.

The patient should receive a sufficient daily dosage of LNA modified antisense oligonucleotide to achieve an effective yet safe intercellular concentrations of combined oligonucleotides. Those skilled in the art should be readily able to derive appropriate dosages and schedules of administration to suit the specific circumstance and needs of the patient.

When a combination of LNA modified antisense oligonucleotide targeting different target sequences are employed, the ratio of the amounts of the different types of LNA modified antisense oligonucleotide may vary over a broad range. According to one preferred embodiment of the invention, the oligonucleotides of all types are present in approximately equal amounts, by molarity.

The effectiveness of the treatment may be assessed by routine methods, which are used for determining whether or not remission has occurred. Such methods generally depend upon some of morphological, cytochemical, cytogenetic, immunologic and molecular analyses. In addition, remission can be assessed genetically by probing the level of expression of one or more relevant genes. The reverse transcriptase polymerase chain reaction methodology can be used to detect even very low numbers of mRNA transcript. For example, RT-PCR has been used to detect and genotype the three known bcr-abl fusion sequences in Ph<1> leukemias. See PCT/US9-2/05035 and Kawasaki et al., Proc. Natl. Acad. Sci. USA 85, 5698-5702 (1988).

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

### EXAMPLE 1

LNA Oligo Mediated In-vivo Downregulation of mRNA Encoded by the FcεR1αgene

Synthesis of Fully Modified LNA Oligomers: Cur0106

(5′-GTCCACAGCAAACAGA-3′)

Assembly was done at a 15 μmol scale on an Expedite™ Nucleic Acid Synthesis System following standard DMT-on procedures except for the use of a 0.1 M solution of the LNA amidites. Oligomerization was performed using double couplings, oxidations, cappings and detritylation times. Universal CPG support (Glen Res.) was used as solid support. Monomers were synthesised according to International Patent Application WO 99/14226 and International Patent Application WO 00/56746. The protecting groups on the bases and cleavage from the resin was done by using concentrated ammonia at 80 deg. for 16 h.

DMT-on purification of the crude oligo was done by HPLC on a reverse phase column ZORBAX 300, C-18, 9, 4 mm×25 cm, flow 3 ml/min, 20-90% acetonitrile gradient in 0.05 M triethylammonium acetate buffer at pH 7.4. The dry purified product was re-suspended in 500 μl 80% acetic acid. This solution was rotor-evaporated and the residue was suspended in 500 μl 10 mM triethyl ammonium acetate buffer and extracted with 3×1 ml diethylether. The aqueous phase was dried under vacuum. The identity of the pure oligo was confirmed by HPLC (>95%), and by ESI-MS: Calcd: 5385.88; Found: 5385.80.

Synthesis of Partly Modified LNA Oligomers: Cur0102

(5′-GTCCAcₛaₛgₛcₛaₛaₛACAGA-3′)

Assembly was done at a 15 μmol scale on an Expedite™ Nucleic Acid Synthesis System following standard DMT-on procedures and using Beaucage reagens as sulphurizing agent. The procedure was modified by using a 0.1 M solution of the LNA amidites. Oligomerization was performed using double couplings, oxidations, cappings and detritylation times. Universal CPG support (Glen Res.) was used as solid support. Monomers were synthesised according to International Patent Application WO 99/14226 and International Patent Application WO 00/56746. The protecting groups on the bases and cleavage from the resin was done by using concentrated ammonia at 80 deg. for 16 h. DMT-on purification of the crude oligo was purified by HPLC on a reverse phase column ZORBAX 300, C-18, 9, 4 mm×25 cm, flow 3 ml/min, 20-90% acetonitrile gradient in 0.05 M triethylammonium acetate buffer at pH 7.4. The dry purified product was re-suspended in 500 μl 80% acetic acid. This solution was rotor-evaporated and the residue was suspended in 500 μl 10 mM triethyl ammonium acetate buffer and extracted with 3×1 ml diethylether. The aqueous phase was dried under vacuum. The aqueous phase was dried under vacuum. The identity of the pure oligo was confirmed by HPLC (>95%), and by ESI-MS: Calcd: 5285.72; Found: 5285.74.

Thermostability of Duplexes Between Cur0102 and 0106 and Their Complementary DNA Oligos

The thermostability of cur0102 and cure 0106 were determined spectrophotometrically using spectrophotometer equipped with a thermoregulated Peltier element. Hybridisation mixtures of 1 ml were prepared containing 100 mM NaCl, 0.1 mM EDTA and 10 mM Na2HPO4, pH 7.0 and equimolar (1.5 μM) amounts of either of the two LNA oligomers and their complementary DNA. The Tm's were obtained as the first derivative of the melting curves and has the following values: Cur0102-77.7° C. and Cur0106>95° C.

Serum Stability Assay

Samples of whole blood were taken from Wistar rats (200 g). The blood samples were centrifuged for 10 minutes at 3500 rpm at room temperature (RT). The supernatant was used in the stability assay. The two isosequential oligomers phosphothioate LNA gab-mer (PS LNA gab-mer [cur0102: 5′-GTCCAcₛaₛgₛcₛaₛaₛACAGA-3′]) and Fully Modified LNA (FM LNA [cur0106: 5′-GTCCACAGCAAACAGA-3′]) were investigated in parallel at a final concentration of 10 μM in rat serum and water. The samples were incubated at 37° C. and 20 μl aliquots were withdrawn at time points 0, 2, 4, 8 and 24 hours, to 7 μl formamide Dye (FD) loading buffer (95% formamide, 0.025% SDS, 0.025 bromophenol blue, 0.025% xylene cyanol FF, 0.025% ethidium bromide and 0.5 mM EDTA; MBI Fermants #R0641) on ice. The samples were stored at −20° C.

Nuclease activity in rat serum was tested by adding DNA oligo cur0209 (5′-gtccacagcaaacaga-3′) at a final concentration of 20 μM. The DNA oligo 0209 was added after 0, 2, 4, 8, and 24 hours, each time to separate tubes of rat serum. All the tubes with rat serum have been incubated at 37° C. from time point zero. At 0, 30 and 60 minutes after the addition of DNA oligo 0209, 10 μl samples were withdrawn to 7 μl formamide Dye (FD) loading buffer (95% formamide, 0.025% SDS, 0.025 bromophenol blue, 0.025% xylene cyanol FF, 0.025% ethidium bromide and 0.5 mM EDTA; MBI Fermants #R0641) on ice. The samples were stored at −20° C.

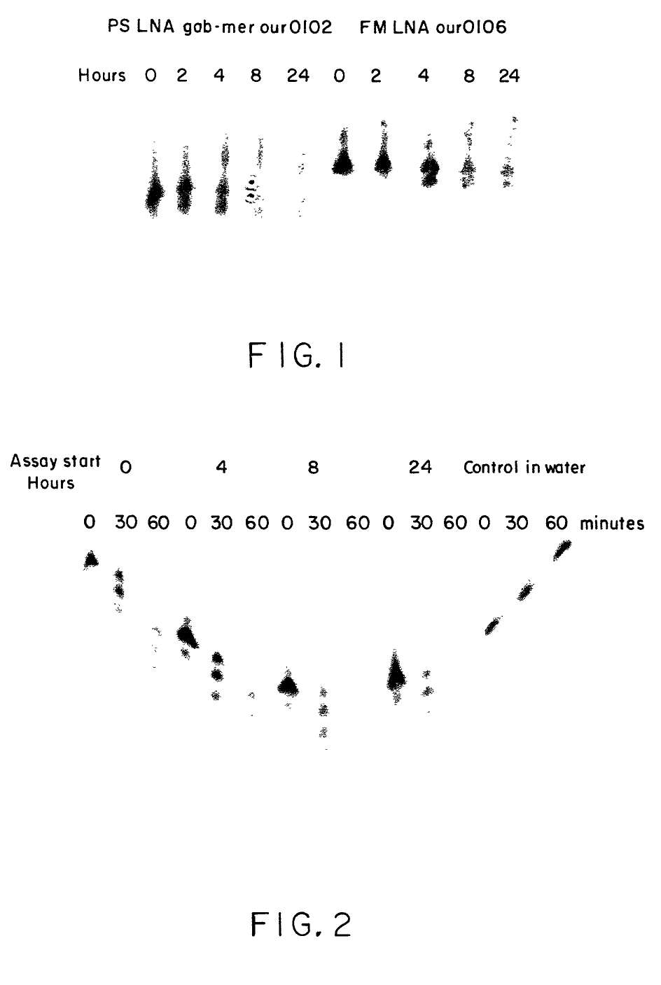

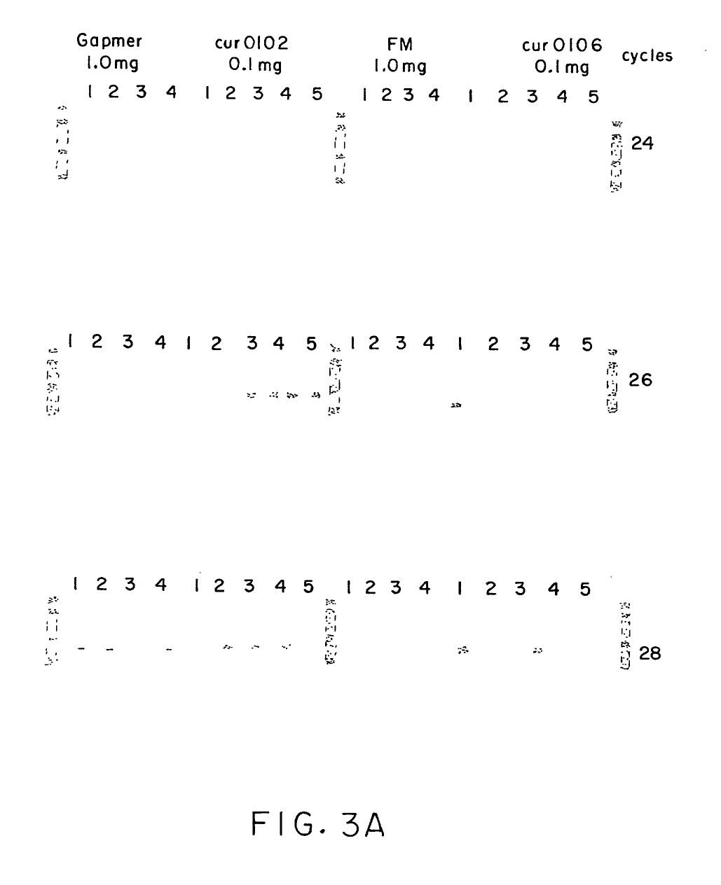

Half the volume of the withdrawn samples from the stability and nuclease activity assays were heated to 95° C. for 2 minutes followed by 2 minutes on ice before the oligomers were separated on a denaturing 13.5% polyacrylamide gel (8 M urea, 3.75% crosslinked [40:1.5]). The 1 mm, 13.5% polyacrylamide gel was runned with 30 watt as limiting parameter for 1 hour and 15 minutes. The gels were stained with SYBR Gold Nucleic Acid Gel Stain (S-11494, Molecular Probes) for visualization of the oligomers. The gels were scanned in a Bio-Rad Molecular Imager FX. The corresponding images of the stability and nuclease activity assays are shown in FIG. 1 and 2.

FIGS. 1 and 2 shows the stability of the phosphothioate LNA gab-mer (cur0102), the Fully Modified LNA (cur0106) and the corresponding DNA oligo in rat serum. Both LNA containing oligomers are relatively stable as judged by the presence of intact full length products at the end of the 24 hour incubation period (FIG. 1). In comparison the isosequential DNA oligo is rapidly degraded by the rat serum as evidenced by the disapperance of essentially all full length products after 60 min (FIG. 2).

FcεR1αmRNA Downregulation and its Effect on the Ability of the Mast Cells to Release Histamine

Animals

Healthy male Whistar rats (19-36) (M&B, Ry, DK) of approximately 0.200 kg were injected 1.5 ml intraperitoneally on day 1 with 1½ ml of isotonic saline containing either 1 or 0.1 mg of either Cur 0102 (gapmer) 5′-GTCCAcₛaₛgₛcₛaₛaₛACAGA-3′, Cur 0106 (FM) 5′-GTCCACAGCAAACAGA -3′ or no oligo (negative control). 4 animals received 1 mg of gapmer, 5 animals received 0.1 mg of gabmer, 4 animals received 1 mg FM, 5 animals received 0.1 mg FM and 3 animals received only isotonic saline.

The injections were repeated on day 4, 7, 10, 13 prior to the sacrifice of the animals on day 15.

The weight of each animal was monitored every third day during the injection period and just before the sacrifice. No abnormal behavior was observed among the animals during the injection period.

Cellular Extractions

The animals were sacrificed and the abdominal fur was removed with a scissor. Approximately 10 ml washing solution (PBS, 0.1% HSA, heparin) was administered to the peritoneal cavity through a cut in the abdominal and the fluid was massaged around in the peritoneal cavity for 90 seconds prior to evaporation to polypropylen tube using a disposable pipette. The suspension was centrifuged at 500 g for 10 minutes and the pellet was washed twice in PBS (0.1% HSA). The cells were subjected to a Alcian blue stain for counting the mastcells. 10 μl cell suspension was diluted in 40 μl 0.1%EDTA (0.9% NaCl), mixed with 1 volume of Basophil counting solution B (cetyl pyridin clorid 380 mg, rathan chlorid 3500 mg, NaCl 4500 mg, Tween 20 1050 mg, Alcian blue 715 mg ad to H2O 500 ml.)(Bie & Bemtsen, Rødovre, DK) and incubated for 5 minutes before counting using a Neubauer improved cytometer.

Functional Analysis for Histamine Release

The histamine release from the peritoneal cells was performed as described by Stahl Skov and colleages (Stahl et al., 1984) using different concentrations of the antibody Anti-rat FcεRI α subunit (cat# 05-0468, Upstate Biotechnology, Lake Placid, N.Y.).

RNA Extractions

Total RNA extractions were performed using TRIzol® Reagent, (cat#15596, Life Technologies, GibcoBRL, Roskilde, DK) Cells washed out from the peritonal cavity of 21 male Whistar rats, were precipitated by centrifugation 500 g, 5 minutes, and the pellet was subjected to lysation in 1 ml Trizol reagent. The suspension was left at room temperature for 5 minutes prior to addition of 0.2 ml chloroform, (cat#C2507E, Labscan Ltd., Dublin, Ireland) followed by vigourous mixing for 15 seconds and incubation for 2-3 minutes at 25° C. The solution was separated in 2 phases by centrifugation at 13,000 rpm in a standard microcentrifuge for 15 minutes at 4° C. The aqueous phase was transferred to a new vail, and RNA was precipitated by 10 minutes incubation at 25° C. with 500 μl isopropanol. RNA was precipitated by centrifugation at 13,000 rpm in a standard microcentrifuge for 15 minutes at 4° C. The supernatant was discarded, and the pellet was washed with 1 ml 70% ethanol, prior to centrifugation for 5 minutes at 7500 rpm in a standard microcentrifuge. The washed pellet was dried and resuspended in Rnase free H₂O by incubation for 10 minutes at 60° C. The quality of the RNA was visualized in a 1% agarose gel (0.5 mg/L ethidium bromide). The concentration and purity was determined by absorption at 260 nm and calculating the ratio A260/A280.

RT-PCR of Rat FcεRI α mRNA

First strand synthesis was performed using Superscript™ I Rnase H⁻ Reverse Transcriptase (cat# 18064-014, Life Technologies, GibcoBRL, Roskilde, DK) 5 μg total RNA from 21 rats was adjusted to 9 μl each with Rnase free H₂O and mixed with 2 μl T25V(pdT)25 10 μM (Display Systems Biotech, cat# 570-100, Vista, Calif., US), 1 μl dNTP mix (10 mM) and incubated 65° C. followed by addition of 4 μl 5× First-Strand buffer [250 mM Tris-HCl, pH 8.3 at room temp, 375 mM KCl, 15 mM MgCl₂], 2 μl DTT (0.1M), 1 μl RNAguard™ Rnase INHIBITOR (33.3 U/ml), (cat# 27-0816-01, Amersham Pharmacia biotech, Hørsholm, DK). The mixture was incubated at 42° C. for 2 minutes prior to addition of 1 μl Superscript II, (200 U/μl) followed by incubation at 42° C. for 50 minutes and heat inactivation of the enzyme at 70° C. for 15 minutes.

PCR of rat FcεRI α amplicon 1 (421 bp) was performed using Platinum® Taq DNA polymerase (cat# 10966-034, Life Technologies, GibcoBRL, Roskilde, DK) cDNA from the first strand synthesis of each of the 21 samples was diluted ten times with Rnase free H₂O.

Reaction Mixture

1.25 μl PCR buffer 10× [200 mM Tris-HCl (pH 8.4), 500 mM KCl]

0.25 μl dNTP mix (10 mM)

0.375 μl MgCl₂ (50 mM)

0.625 μl forward primer 10 μM (5′-TGTGAGTGCCACCATTCAAGACAGT-3′)

0.625 μl reverse primer 10 μM (5′-GTCCACAGCAAACAGAATCACCGCC-3′)

0.25 μl cDNA

0.125 μl Platinum Taq polymerase (5 U/μl)

H₂O ad to 12.5 μl

PCR reaction: (Thermocycler, GeneAmp PCR system 9700, Applied Biosystems) 94° C. 2 minutes, [94° C. 30 seconds, 72° C.* 30 seconds]×A, 72° C. 7 minutes.

* decrease of 0.5° C. per ongoing cycle.

The PCR reactions were terminated after different number of cycles, A A=24, 26, 28, 30, 32, 34

5 μl PCR-product was loaded on a 1% agarose gel, and visualized with Ethidium Bromide (0.5mg/L), using 100 bp DNA ladder (cat#15628-019, Life Technologies, GibcoBRL, Roskilde, DK) as molecular weight standard. Gels were scanned with Molecular Imager Fx (Biorad).

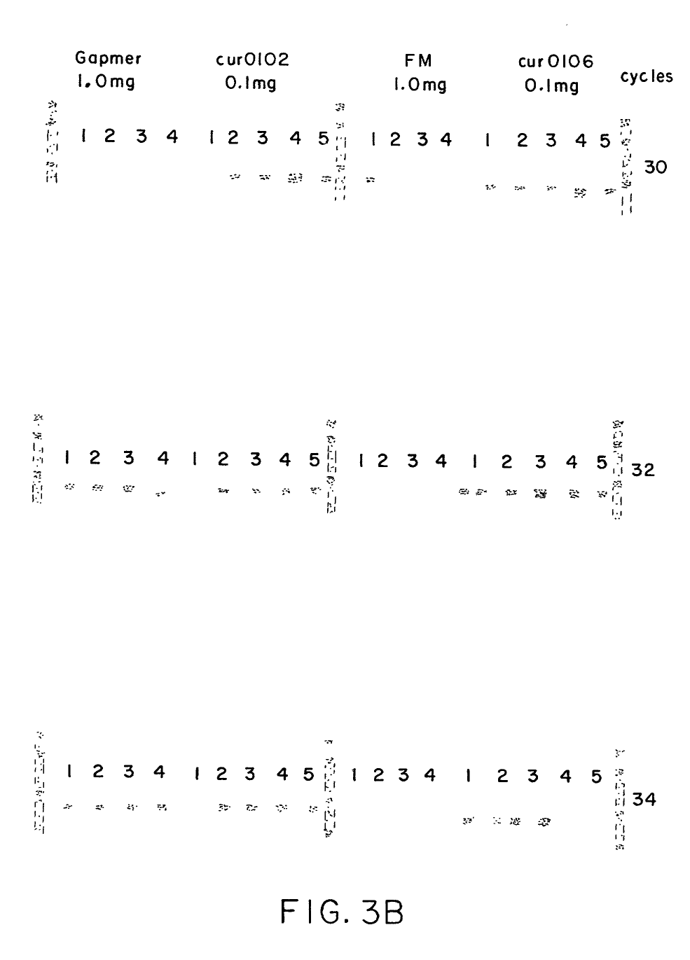

As indicated in, FIG. 3, and summarized in Table 6, amplicons appears earlier in the PCR reactions conducted on material from rats treated with low doses of either of the two LNAs than on material obtained from rats treated with high doses of the LNAs, indicating that the higher dose of LNA reduces the cellular steady state level of the target mRNA more efficiently than the lower doses.

TABLE 6
RT-PCR analysis of the total RNA from peritoneal cells.
(Indication of the PCR-cycle that visualized the rat FcεRI α amplicon 1 (421 bp))

| Compound | Rat # | | | | | | | | | | | | | | | | | |
|---|---|---|---|---|---|---|---|---|---|---|---|---|---|---|---|---|---|---|
| injected | 19 | 20 | 21 | 22 | 23 | 24 | 25 | 26 | 27 | 28 | 29 | 30 | 31 | 32 | 33 | 34 | 35 | 36 |
| Gapmer high | 28 | 28 | 28 | 28 | | | | | | | | | | | | | | |
| (1 mg) | | | | | | | | | | | | | | | | | | |
| Gapmer low | | | | | — | 26 | 26 | 24 | 26 | | | | | | | | | |
| (0.1 mg) | | | | | | | | | | | | | | | | | | |
| FM high (1 mg) | | | | | | | | | | 34 | — | — | — | | | | | |
| FM low (0.1 mg) | | | | | | | | | | | | | | 26 | 28 | 26 | 28 | 30 |

# Italics. PCR-cycle w. visible product
Gapmer. Cur 0102 LNA/DNA w. partiel PS backbone
FM. Cur 0106 Fully modified LNA
— No amplicons detected
Reference: Stahl,SP, S Norn, B Weeke, 1984, A new method for detecting histamine release: Agents Actions, v. 14, p. 414-416.

As shown in Table 7 the histamine release from cells obtained from antisense treated rats is statistically lower than the corresponding release from Mock treated rats (injected with isotonic saline). The control antibody (isotype), which do not recognize the FcεR1 receptor, do not induce a histamine release in any of the cells tested, substantiating that the differences observed in histamine release between the Mock and antisense cells are due to differences in the number of functional receptors on these cells.

TABLE 7

| | Dose | Antibody | 1:10 dil. | 1:20 dil. | 1:40 dil. |
|---|---|---|---|---|---|
| Cur0106 | 1 mg | FcεR1 | 30% +/− 7.0 | 0.75% +/− 1.5 | 0.75% +/− 1.5 |
| Cur0106 | 1 mg | Isotype | 0.75% +/− 71.5 | 0.75% +/− 1.0 | 0.75% +/− 1.5 |
| Cur0106 | 0.1 mg | FcεR1 | 35% +/− 6.0 | 3.0% +/− 3.5 | 1.5% +/− 0.5 |
| Cur0106 | 0.1 mg | Isotype | 0.8% +/− 1.1 | 1.6% +/− 3.6 | 1.0% +/− 0.75 |
| Cur0102 | 1 mg | FcεR1 | 37% +/− 8.0 | 2.8% +/− 3.0 | 1.0% +/− 0.75 |
| Cur0102 | 1 mg | Isotype | 23% +/− 4.5 | 2.0% +/− 4.0 | 1.0% +/− 0.75 |
| Cur0102 | 0.1 mg | FcεR1 | 43% +/− 1.0 | 4.0% +/− 3.5 | 1.2% +/− 0.6 |
| Cur0102 | 0.1 mg | Isotype | 1.6% +/− 2.0 | 1.4% +/− 3.0 | 1.0% +/− 0.75 |
| Mock | — | FcεR1 | 56% +/− 17 | 13% +/− 3.0 | 4.6% +/− 3.0 |
| Mock | — | Isotype | 2.7% +/− 4.6 | 0.0 | 1.5% +/− 0.75 |

All documents mentioned herein are fully incorporated herein by reference in their entirety.

What is claimed is:

1. A method of modulating expression of a gene involved in malignant cell growth, comprising contacting the gene or RNA from the gene with an oligonucleotide that comprises one or more LNA units, whereby gene expression is modulated.

2. The method of claim 1 wherein contact with the LNA oligonucleotide results in inhibition of expression of the gene.

3. A method of modulating expression of an oncogenc, tumor suppressor gene, a DNA repair gene, an MMP gene, a gene encoding a multidrug transporter protein, or a gene involved in the signal transduction pathway regulating cell growth, comprising contacting the gene or RNA from the gene with an oligonucleotide that comprises one or more LNA units, whereby gene expression is modulated.

4. The method of claim 3 wherein contact with the LNA oligonucleotide results in inhibition of gene expression.

5. The method of any one of claims 1 through 4 wherein the gene comprises at least a portion of a sequence identified in table 1 above.

6. The method of claim 2 or claim 4 wherein the LNA oligonucleotide hybridizes with messenger RNA of the gene to inhibit expression thereof.

7. A method of treating a mammal suffering from or susceptible from malignant cell growth, comprising:
administering to the mammal an effective amount of an oligonucleotide that comprises one or more LNA units.

8. The method of claim 7 wherein the malignant cell growth comprises a solid tumor or a leukemic malignancy.

9. The method of claim 7 or claim 8 wherein the malignant cell growth is present in a lung, liver, stomach, intestine, bowel, prostate, brain, testes or ovaries of the mammal.

10. The method of any one of claims 7 through 9 wherein the mammal suffers from undesired expression of an oncogene, a tumor suppressor gene, a DNA repair gene, an MMP gene, a gene encoding a multidrug transporter protein, or a gene involved in the signal transduction pathway regulating cell growth.

11. The method of any one of claims 7 through 10 wherein the mammal suffers from undesired expression of at least a portion of a sequence identified in table 1 above.

12. The method of claim 10 or claim 11 wherein the administered LNA oligonucleotide hybridizes with messenger RNA of the gene or sequence to inhibit expression thereof.

13. A method of modulating expression of a gene associated with an inflammatory disease, comprising contacting the gene or RNA from the gene with an oligonucleotide that comprises one or more LNA units, whereby gene expression is modulated.

14. The method of claim 13 wherein contact with the LNA oligonucleotide results in inhibition of gene expression.

15. The method of claim 13 or claim 14 wherein the gene comprises a CD marker gene, a gene encoding an adhesion molecule, a gene encoding a chemokine or chemokine receptor, a gene encoding interleukin or interleukin receptor, or a gene encoding an immuoglobulin, an immunoglobulin receptor, or a subunit of an immunoglobulin.

16. The method of any one of claim 13, 14 or 15 wherein the gene comprises a gene encoding IgE, FcεRIα, IgG, IgA1, IgA2, IgM, IgD, a gene encoding their corresponding receptors or a gene encoding their subunits.

17. A method of any one of claim 13, 14 or 15 wherein the gene comprises at least a portion of a sequence identified in tables 2, 3, 4 or 5 above.

18. The method of any one of claims 13 through 17 wherein the administered LNA oligonucleotide hybridizes with messenger RNA of the gene or sequence to inhibit expression thereof.

19. A method of treating a mammal suffering from or susceptible from an inflammatory disease or disorder, comprising:
administering to the mammal an effective amount of an oligonucleotide that comprises one or more LNA units.

20. The method of claim 19 wherein the mammal suffers from undesired expression of a CD marker gene, a gene encoding an adhesion molecule, a gene encoding a chemokine or chemokine receptor, a gene encoding interleukin or interleukin receptor, or a gene encoding an immuoglobulin, an immunoglobulin receptor or an immunoglobulin subunit.

21. The method of claim 19 or 20 wherein the mammal suffers from undesired expression of a gene encoding IgE, FcεRIα, IgG, IgA1, IgA2, IgM, IgD a gene encoding their corresponding receptors, or a gene encoding their subunits.

22. A method of any one of claim 19 or 20 wherein the mammal suffers from undesired expression of at least a portion of a sequence identified in tables 2, 3, 4 or 5 above.

23. The method of claim 21 or 22 wherein the administered LNA oligonucleotide hybridized with messenger RNA of the gene or sequence to inhibit expression thereof.

24. The method of any one of claims 1 through 23 wherein the LNA oligonucleotide comprises from about 8 to about 60 base units.

25. The method of any one of claims 1 through 24 wherein the LNA oligonucleotide comprises from about 10 to about 40 base units.

26. The method of any one of claims 1 through 25 wherein the LNA oligonucleotide comprises one or more units of formula 1a or 1b as defined above.
